(19) United States
(12) Patent Application Publication
(10) Pub. No.: US 2007/0208103 A1
(43) Pub. Date: Sep. 6, 2007

(54) ANTIMICROBIAL PROSTHETIC DENTAL ELEMENT AND ITS PRODUCTION METHOD

(75) Inventors: Pierre-Luc Reynaud, Vaulnaveys Le Haut (FR); Manh-Quynh Chu, Fontanil Cornillon (FR); Gerard Piet, Saint Pierre D'Allevard (FR); Laurent Bedel, Quaix en Chartreuse (FR)

Correspondence Address: HESLIN ROTHENBERG FARLEY & MESITI PC, 5 COLUMBIA CIRCLE, ALBANY, NY, 12203, US

(73) Assignee: Recherches Et Techniques Dentaires - R.T.D., Saint Egreve (FR)

(21) Appl. No.: 11/680,829

(22) Filed: Mar. 1, 2007

(30) Foreign Application Priority Data
Mar. 2, 2006 (FR) 0650740

(51) Int. Cl. A61K 6/083; A61K 33/34; A61K 33/38

(52) U.S. Cl. 523/115; 424/618; 424/638; 433/217.1

(57) ABSTRACT

Prosthetic dental element is obtained by machining a rod of composite material made of fibres embedded in a matrix containing resin. All or part of a surface of the machined rod is coated with at least one layer containing at least one agent with an antimicrobial effect.

### CROSS REFERENCE TO RELATED APPLICATIONS

This application is related to commonly assigned, concurrently filed U.S. application Ser. No. ______ (Docket 1759.258) and Ser. No. ______ (Docket 1759.259) and claims priority from French patent application 0650740 filed on Mar. 2, 2006.

### BACKGROUND ART

This invention concerns a prosthetic dental element made of composite material, said element having antimicrobial properties. The invention also concerns the production method for said prosthetic dental element.

“Prosthetic dental element” notably, but not restrictively, designates pegs and bridge reinforcements, excluding implants, which are elements designed to be inserted directly into the maxillary bone.

In the rest of the description, the invention is more particularly described in relation to a dental peg made of a composite material.

Dental pegs are used for the reconstitution of pulpless teeth. A distinction is made between two types of pegs, respectively:
metal or ceramic pegs, and
composite pegs.

Metal pegs are usually made of stainless steel. Their main disadvantage is that they are subject to corrosion phenomena. Moreover, they have a transversal modulus of elasticity that is different from that of the dentine, leading to peg separation over time.

To solve these problems, pegs made of composite materials have been proposed, notably such as those described in the Applicant's document EP-A-432 001. In practice, these pegs are constituted of long, unidirectional fibres made of glass or carbon and embedded in a thermosetting resin matrix. In general, the proportion of long fibres, whatever the material selected, accounts for 55 to 70% of the volume of the peg, the complement to 100% being occupied by the matrix.

To maintain the peg in the root, it must be glued and then sealed into said root. The practitioner then fills in the remaining spaces with a composite paste. The prosthesis or crown then covers the whole and is held in place by adding additional cement, if necessary accompanied by an adhesive.

Despite the care given to the various clinical steps in reconstituting the tooth, we can sometimes observe a recurrence of caries leading to, at best, a loosening of the prosthesis requiring complete retreatment of the root or, at worst, to the extraction of the tooth.

These recurring caries are due to an infiltration of pathogenic germs, such as Streptococcus mutans, Enterococcus faecalis, or even, in some cases, Staphylococcus aureus. Such infiltration may be due to loss of marginal seal between the prosthesis, the dentine and the filling material. Sources of bacteria may also subsist when dentine etching is poorly executed and lets debris appear. Germs can also take advantage of natural obstacles such as undercuts, working as receptacles.

To solve this problem, several solutions have been developed, such as antimicrobial adhesives or cements, but also etching solutions.

Thus, document U.S. Pat. No. 5,385,728 describes an acid etching composition containing benzalkonium chloride as an antimicrobial agent.

Document U.S. Pat. No. 5,968,253 discloses a calcium phosphate-based dental cement which may contain an antimicrobial agent in the form of an antibiotic or an antiamebic.

Document U.S. Pat. No. 6,326,417 concerns an antimicrobial dental composition used as a restorative adhesive, cement or composite paste containing compounds of salicylic acid or sulfanilamide, possibly in derived form.

Documents U.S. Pat. No. 5,408,022, U.S. Pat. No. 5,494,987 and U.S. Pat. No. 5,733,949 describe polymer-based compositions with antimicrobial properties after polymerisation, used to produce artificial skin, catheters, contact lenses as well as dental products, such as adhesives, resins and composites. The antimicrobial agent is a polymerisable monomer, whose active group is based on a quaternary salt (chlorine or bromine).

Document U.S. Pat. No. 6,355,704 describes an antimicrobial dental adhesive with two components. One of the components is an antimicrobial polymerisable monomer with a cationic group selected from the ammonium, pyridinium and phosphonium bases.

Document U.S. Pat. No. 6,924,325 describes an antimicrobial dental composition which can be used as a restorative endodontic, orthodontic or prosthetic composition. This composition contains as its active ingredient a ceramic glass/zinc-silver powder or a silver-based zeolite powder.

Products of other types than the aforementioned with antimicrobial properties have been developed. These are notably compositions used for whitening teeth, as described in documents U.S. Pat. No. 5,985,249, U.S. Pat. No. 6,036,943, U.S. Pat. No. 6,309,625 and U.S. Pat. No. 6,368,576, in which the antimicrobial agent is selected from among chlorhexidine, benzoate derivatives, tetracycline or fluorinated compounds.

Document U.S. Pat. No. 6,365,130 describes a chewing gum containing antimicrobial ceramic particles based on metal cations.

Document U.S. Pat. No. 6,267,590 deals with a bracket and an orthodontic wire treated by the deposition of a silver zeolite ceramic polymer.

Document U.S. Pat. No. 6,113,993 deals with a method for treating a dental implant, said treatment consisting in applying to the surface of the implant, a calcium phosphate-type binding agent designed to encourage bone growth around the implant. Once the treatment has been applied, it is proposed that the implants should be soaked in an antimicrobial agent solution before implanting. After implanting, the calcium phosphate will be released at the same time as the agent to encourage growth and/or prevent infection.

In another field, document XP002295856 describes medical devices such as catheters coated with silver in the form of metal ions. This study demonstrates unequal antimicrobial efficacy depending on the devices coated.

To the Applicant's knowledge, no solution has been proposed to make a composite prosthetic element such as a peg, antimicrobial, for which there are a certain number of constraints, notably:
preserving the oral flora,
maintaining mechanical characteristics,
providing a sufficient level of transparency for the transmission of light beams from a curing lamp.

### BRIEF SUMMARY OF THE INVENTION

The Applicant has developed a prosthetic element, all or part of the surface of which is coated with at least one layer containing an antimicrobial agent.

In other words, the subject of the invention is a dental prosthetic element obtained by machining a section of composite material made of fibres embedded in a matrix, notably containing resin.

This prosthetic element is characterised in that it is coated, over all or part of its surface, with at least one layer containing at least one agent with an antimicrobial effect.

In one particular embodiment, the prosthetic element is a peg. In practice, the peg comprises reinforcement fibres embedded in a matrix containing resin. These may be glass, carbon, ceramic or quartz fibres.

In practice, the antimicrobial agent is a metal. Advantageously, the antimicrobial agent is selected from the group including silver and copper.

To improve adhesion and therefore to avoid release of the antimicrobial agent from the peg or more generally from the prosthetic element, the coating also contains at least one non-organic substance such as, for example, a metal oxide. In this situation, the metal is applied to the surface of the prosthetic element, possibly in a mixture with the metal oxide. Advantageously, the metal oxide is selected from the group including aluminium oxide, barium oxide, strontium oxide and zirconium oxide.

In practice, the coating containing the antimicrobial agent has a thickness between 0.01 μm and 5 μm, advantageously 0.25 μm, this thickness being determined at the most regular contour of the peg. For a lesser thickness, the antimicrobial properties are not as good. A greater thickness is too much to allow satisfactory insertion of the peg or leads to aesthetic problems.

Moreover, and according to another characteristic, the antimicrobial agent accounts for 0.001% to 1% by weight, advantageously 0.25% by weight, of the coating. In relation to the prosthetic element, and notably the peg, the antimicrobial agent accounts for 0.001% to 5% by weight, advantageously 0.05%. Outside this range, the antimicrobial effect is insufficient or no better.

In one advantageous embodiment, the prosthetic element is not only antimicrobial but also radiopaque.

To make the product visible to X rays, the prosthetic element contains a radiopaque substance.

Those skilled in the art know of several ways of making the product radiopaque.

In a first embodiment, at least one radiopaque product is incorporated into the fibre. In this case, the fibres used may have, for example, a calcium oxide or zirconium oxide base.

In a second embodiment, at least one radiopaque substance is incorporated into the matrix.

A third possibility, which may be combined with one or the other, or both aforementioned solutions, is to incorporate the radiopaque agent into the surface coating. Advantageously, the same metal oxide will be used both to provide radiopacity to the prosthetic element and to improve adhesion of the antimicrobial agent.

In practice, the radiopaque substance is selected from the group including metal oxides such as aluminium oxide, barium oxide, strontium oxide, zirconium oxide or fluorinated compounds, such as ytterbium and yttrium, alone or in mixtures. Zirconium oxide is preferred when seeking to obtain both properties, adhesion of the antimicrobial agent and radiopacity, respectively.

To encourage the bonding, and then the sealing of the peg in the roots using a composite cement, it is preferable to treat the peg with a binding agent, notably silane or an adhesive. In practice, the practitioner applies the silane extemporaneously to the surface of the peg, i.e. when it is placed in the root. Now, it is known that the silanisation of a composite material is usually mediocre due to the absence of effective silane binding.

The Applicant has observed that, quite surprisingly, the addition of the non-organic substance blocking the release of the antimicrobial agent in the coating containing said antimicrobial agent also provides improved silane binding to the peg for its gluing. The pegs can thus be supplied to the practitioner in silanised form, so that the practitioner does not have to perform extemporaneous preparation. In other words, and in an advantageous embodiment, the coating containing the antimicrobial agent also contains a binding agent such as a silane, for example.

In practice, the silane is selected from the group including 3-(trimethoxysilyl)propyl methacrylate, vinyltrimethoxysilane, 3-(glycidyloxy)propyl trimethoxysilane and 3-(trimethoxysilyl)propyl methacrylate.

The invention also concerns a method for producing the prosthetic element, notably the aforementioned peg.

According to this process, a section of composite material is first prepared containing fibres embedded in a matrix notably containing resin.

When the prosthetic element is a peg, the section contains reinforced fibres embedded in a resin matrix, for example an epoxy resin.

In practice, the fibres are glass fibres, possibly radiopaque, ceramic fibres or quartz fibres.

Once the section has been obtained, it is machined to obtain the desired shape of the prosthetic element.

The surface of the prosthetic element is then coated with at least one layer containing at least one agent with an antimicrobial effect.

In a particular embodiment, the coating also contains a non-organic substance, such as a metal oxide. This non-organic substance performs a first function that is to improve adhesion of the metal to the surface of the prosthetic element. When the non-organic substance is a potentially radiopaque metal oxide, it performs a second function which is to make the prosthetic element radiopaque. The various components of a given coating may be applied simultaneously or successively.

In an additional step, the prosthetic element is silanised, i.e. at least one binding agent such as a silane is applied to all or part of the coating containing an antimicrobial agent. The coating containing the antimicrobial agent and possibly a non-organic substance is applied using the technique called VPD (vapour-phase deposition), advantageously by magnetron cathode sputtering or by CVD (Chemical Vapour Deposition) techniques. On the other hand, the binding agent is in practice applied by simple soaking, notably in a silane solution.

### DETAILED DESCRIPTION

The invention and the resulting advantages will be made clear by the following non-restrictive examples of embodiments.

### Example 1

The pegs are obtained after machining of a section containing E-glass fibres representing a volume of 60% embedded in a epoxide resin matrix with a Bisphenol A base, said matrix representing 40% by volume and not containing any radiopaque fillers.

Deposition of a Coating Containing Silver and Zircon on the Surface of Dental Pegs

The deposits are formed simultaneously by magnetron cathode sputtering from a zirconium target and a silver target. The pegs attached to a metal support are positioned 10 cm from the targets. Before deposition, a 4.10⁻³ Pa vacuum is formed in the reactor. Argon and oxygen are introduced during the deposition step. The targets are bombarded using a 700 W and 300 W pulsed DC discharge, respectively, for the zirconium target and the silver target under 1 Pa pressure.

The coating containing the silver covers the entire surface of the pegs, except for a 2- to 3-mm length which the practitioner will cut off when putting it in place.

The thickness of the deposit obtained is approximately 0.250 μm (weak concentration of silver) and it has no influence on the size characteristics of the peg. The space for gluing and sealing is usually at least 20 μm on the radius.

During this deposition, 8-mm diameter samples of the same material as that used in machining the pegs were also treated.

Antimicrobial Activity:

Three coated 8-mm diameter samples underwent antimicrobial activity tests at the same time as three untreated samples used as controls.

The test method is inspired by Japanese Industrial Standard JIS Z 2801 “Antimicrobial product-Test for antimicrobial activity and efficacy”.

The bacterial strain used was Staphylococcus aureus.

All samples were incubated for 24 h at 37° C. The populations of Staphylococcus aureus were then counted. The antimicrobial activity of the sample is equal to the difference of the average of the three control samples minus the average of the three samples tested. Antimicrobial activity is equal to the log of the difference between the number of bacteria counted in the control sample minus the number of bacteria counted in the sample tested.

The samples from this example totally phagocyted all of the bacteria, as 0 germs were counted after 24 h of incubation.

Bactericidal activity was:
5-5.28 log on Staphylococcus aureus,
4.42 log on Streptococcus mutans,
5.02 log on Enterococcus faecalis.
   Mechanical Properties:

The pegs were sunk into a bronze support and inclined at 45°. Force was applied at a constant speed of 1 mm/minute by a push-pull machine. The maximum load at break was then recorded.

Results:

Untreated control pegs: 94 N±4N maximum load

Treated pegs: 95 N±6N maximum load

The treated pegs in this example allowed curing of the adhesive as did the control pegs.

Gluing

The pegs having received an antimicrobial treatment were then soaked in a solution containing silane MEMO for 15 minutes. They were then dried in a drier to evaporate the solvent and water.

MEMO Solution:

Ethanol solution containing approximately 10% acetic acid acidified water
MEMO: 3-(trimethoxysilyl)propyl methacrylate 5% by weight

The pegs were then tested using the Applicant's so-called “push-out” method. The shear adhesion value is expressed in MPa.

The gluing results obtained with the pegs produced according to this example were improved: 32 MPa rather than 26.9 MPa with an adhesive and 32 MPa rather than 23 MPa without adhesive. The silanised pegs without antimicrobial treatment only reached a value of 29 MPa.

The gluing or sealing protocol was reduced by one step.

Indeed, it is no longer necessary to apply an adhesive coating to the peg and then to light cure it.

### Example 2

Pegs identical to those in the previous example were treated under the same conditions, except for the exposure time, which was multiplied by three.

The thickness of the deposit obtained was approximately 0.25 μm, close to the previous test but the silver content was multiplied by ten.

The treated samples were subjected to the antimicrobial activity described above. The bactericidal activity was 5.19 log on Staphylococcus aureus.

The results of the tests for fracture at 45° and light transmission were close to the previous results and to those of the control pegs.

Concerning the results of the gluing tests, the value obtained was 29.7 MPa rather than the 32 MPa in the previous example.

They confirmed that the antimicrobial deposit provided values at least equal to those of the untreated pegs and especially that this coating improved adhesion compared to that of a peg glued with a conventional adhesive (26.9 MPa) and with no adhesive (23 MPa).

The pegs treated by this method not only provide sufficient antimicrobial activity, without undesired secondary toxicity, while preserving a strong, or even improved, adhesive property and reducing the clinical protocol by one step.

### Example 3

Pegs identical to those in example 2 were treated under the same conditions (sputtering time multiplied by ten) using copper.

The thickness of the deposit obtained was approximately 0.25 μm, similar to the previous test.

The treated samples were subjected to the antimicrobial activity described previously.

Their bactericidal activity was 2.84 log on Streptococcus mutans.

The results of the tests for fracture at 45° and light transmission were close to the previous results and to those of the control pegs.

Concerning the results of the gluing tests, the value obtained was 34.9 MPa.

They confirmed that the antimicrobial deposit provided values at least equal to those of the untreated pegs and especially that this coating improved adhesion compared to that of a peg with a conventional adhesive (26.9 MPa) and with no adhesive (23 MPa).

This invention presents a great number of advantages, including:
localised antimicrobial action without release of elements which could destroy the oral flora or disseminate secondary elements throughout the organism,
preservation of mechanical properties (deposition technique, its thickness and nature had no negative influence on the peg),
preservation of the transparency of the peg made of a fibred composite material allowing the transmission of light beams from the curing lamp,
respect of the peg's initial surface roughness,
compatibility with silanisation,
preservation or even improvement of the adhesion values between the peg and the cement without using an adhesive or primer.

1/ Prosthetic dental element comprising a machined rod of composite material comprising fibres embedded in a matrix containing resin, wherein all or part of a surface of the machined rod is coated with at least one layer containing at least one agent with an antimicrobial effect.

2/ Prosthetic element as claimed in claim 1, wherein the antimicrobial agent comprises a metal.

3/ Prosthetic element as claimed in claim 2, wherein the metal comprises silver or copper.

4/ Prosthetic element as claimed in claim 1, wherein the layer also contains a non-organic substance.

5/ Prosthetic element as claimed in claim 4, wherein the non-organic substance comprises a metal oxide.

6/ Prosthetic element as claimed in claim 1, wherein the layer containing at least one agent with an antimicrobial effect has a thickness between 0.01 μm and 5 μm.

7/ Prosthetic element as claimed in claim 1, wherein the layer containing at least one agent with an antimicrobial effect also contains at least one radiopaque substance.

8/ Prosthetic element as claimed in claim 7, wherein the radiopaque substance is selected from the group consisting of metal oxides and fluorinated compounds.

9/ Prosthetic element as claimed in claim 1, wherein the layer also contains a silane.

10/ Method for producing a composite prosthetic dental element according to which:
a rod is prepared containing fibres embedded in a matrix containing resin,
the rod is machined to a desired shape of the prosthetic element, and
a layer containing at least one agent with an antimicrobial effect is applied to the machined rod.

11/ Method according to claim 10, wherein the layer containing at least one agent with an antimicrobial effect also contains a non-organic substance.

12/ Method according to claim 10, wherein the layer containing at least one agent with an antimicrobial effect also contains a radiopaque substance.

13/ Method according to claim 12, wherein the radiopaque substance is selected from the group consisting of metal oxides and fluorinated compounds.

14/ Method according to claim 10, wherein the layer containing at least one agent with an antimicrobial effect is applied by vapour-phase deposition.

15/ Method according to claim 10, wherein, once the layer containing an antimicrobial agent has been applied, the coated machined rod is silanised.

16/ Prosthetic element as claimed in claim 1, wherein the coated machined rod comprises a post.

17/ Prosthetic element as claimed in claim 8, wherein the metal oxides consist of aluminium oxide, barium oxide, strontium oxide, zinc oxide, and zirconium oxide, and the fluorinated compounds consist of ytterbium and yttrium.

18/ Method according to claim 13, wherein the metal oxides consist of aluminium oxide, barium oxide, strontium oxide, zinc oxide, and zirconium oxide, and the fluorinated compounds consist of ytterbium and yttrium.

19/ Method according to claim 14, wherein said vapour-phase deposition comprises magnetron cathode sputtering or chemical vapour deposition.
